(19) United States
(12) Patent Application Publication
(10) Pub. No.: US 2004/0071590 A1
(43) Pub. Date: Apr. 15, 2004

(54) Fixed vacuum-insulated saturated steam autoclave

(76) Inventor: Melvyn Lloyd Sawyer, Vancouver (CA)

Correspondence Address: Melvyn L. Sawyer, 321 Medical Building, 650 West 41st. Ave., Vancouver, BC, V5Z 2M9, CA

(21) Appl. No.: 10/270,428

(22) Filed: Oct. 15, 2002

(51) Int. Cl. A61L 2/08; A01N 1/00

(52) U.S. Cl. 422/26; 422/33; 422/295

(57) ABSTRACT

This invention relates to table top steam sterilizers, wherein a novel method of heat conservation within the sterilizer, using fixed vacuum chamber walls, is described. As well, the temperature of the sterilizer is more accurately regulated and stabilized by power application to a single heating coil with the use of a proportional controller, and by use of temperature, pressure and humidity sensors. The method also relates to a post-sterilization moisture evacuation process using a venturi. When this method is employed, the time required for the sterilization process is reduced.

### FIELD OF THE INVENTION

This invention relates to table top steam sterilizers, commonly called autoclaves. In particular, it describes a new design for autoclaves such that heat conservation and steam saturation are ensured during the sterilization process.

### BACKGROUND OF THE INVENTION

Table top steam sterilizers, or autoclaves, are widely used for the sterilization of instruments and other articles used in the medical, dental and veterinary disciplines. Contents of autoclaves, when exposed to the proper conditions of temperature, time and pressure within a saturated steam environment will be sterile, as all spores, viruses, and microorganisms will be destroyed during this process under the proper conditions.

An autoclave sterilizer is typically portable, of small tabletop cabinet size with a single door and latch assembly. Pre-cleaned articles to be sterilized are placed into the autoclave and the door is closed and latched shut. The autoclave operates by evaporating, during each use cycle, a small amount of water released from its self-contained reservoir into its preheated chamber. The chamber is heated by the direct application of heating elements to the outer bottom surface of the chamber. As the water in the chamber is heated to the boiling point, steam is formed. The steam, being lighter than air rises to the top of the chamber, and thus cooler air is displaced by gravity to the bottom of the chamber. This cooler, unsaturated air then evacuates through an open valve. Once the temperature of the chamber reaches a preset value, the chamber is deemed to be saturated with steam and the open valve closes. Pressure and temperature then build in the closed chamber until a predetermined temperature and pressure level is reached. These levels are then held for a prescribed period of time to enable complete destruction of microorganisms, spores and viruses inside the chamber and on the contents therein, rendering a sterile condition. Steam is then released from the chamber and the contents of the chamber are removed.

Table top portable autoclaves essentially are heated pressure vessels that generate their own steam. This is in contrast with the larger fixed hospital type steam sterilizers that are fed steam from a separate steam source. Prior to the injection of steam into the chamber, the air from its closed chamber is evacuated by being subjected to a vacuum.

There are certain well-known and previously described problems that occur with autoclaves. The sterilization chamber, or pressure vessel, must be free of air and filled with saturated steam during the sterilization cycle. Failure to do so could result in pockets of air remaining in the sterilizer, resulting in cold spots where articles would not be subjected to sterilizing conditions. As well, water condensation could occur inside the chamber, also potentially entrapping microorganisms resulting in sterilization failure. Suggested solutions to these problems have included the use of a pre-sterilization vacuum applied to the chamber to remove air prior to steam injection, as well as using sophisticated temperature and pressure measurements conforming to conventional pre-set values which assume air removal from the chamber. The use of a vacuum pump to evacuate air from the chamber adds additional expense and increased potential for mechanical breakdown of the autoclave. The use of temperature and pressure sensors that are controlled by a microprocessor to respond to a predetermined set values also add additional expense to the autoclave. Also, relying on preset and predetermined temperature and pressure values does not take into account variations in atmospheric pressure that exist when the autoclave is operated at altitudes other than sea level.

Another problem that occurs with autoclaves is that it becomes difficult to precisely control the temperature of the chamber, whether the technique of steam injection or steam generation within the chamber via direct application of heating elements are used. Precise temperature control is not only necessary for the assurance of sterility, but it is also critical to ensure that temperatures do not exceed the maximum sterilization temperature that manufacturers recommend for certain temperature sensitive articles. The ability to control temperatures to +/−2 degrees in a pressurized chamber at about 270 degrees Fahrenheit (132 degrees Centigrade) is extremely difficult. This difficulty can be due to heat loss through the chamber wall, the residual heat capacity of the sterilizer's heating elements as they are intermittently turned on and off during the sterilization cycle, or due to a rapid rise in the chamber temperature after injection of steam.

In the case of sterilizers that generate their own steam by heating water in the chamber via a direct application of a heating element to the chamber, such heating elements generally have an on/off application of power. During the sterilization cycle, when the sterilizer cools below the set-point, the heating element is turned on at maximum power. Once the temperature reaches the set point, the power to the element is turned off. This difference in temperature is known as the hysteresis. However, due to the on/off application of power, and the residual heat capacity of the heating element, temperature fluctuation inside the sterilizer is generally greater than the hysteresis. When this occurs, chamber temperature overshoots the set point, and sterilization temperatures can exceed manufacturers maximum sterilization temperature tolerances for instruments.

Feathers et al. (U.S. Pat. No. 5,164,161) discusses the use of a proportional controller which applies heat to a number of heaters placed on a large, 58 kilogram sterilizer chamber. This controller measures the temperature differential between various areas on the chamber wall surface and a set point predetermined by a chosen sterilization cycle. The controller applies electricity, and thus heat, proportionally to the surfaces that require an increased amount of heat. This size of sterilizer requires a prolonged warm-up phase, where maximum power is directed to all the heaters. As the described sterilizer also employs an optional unsaturated chemical sterilization cycle, where the temperature inside the chamber is not necessarily constant throughout, it is important to have regional control over the heating process.

In his discussion of steam injection type sterilizers, it has been suggested by Breach (U.S. Pat. No. 5,858,304) that a vacuum jacket surround a portion of such a steam injection-type sterilizer. A large open space is created between the outer wall of a rectangular shaped sterilizer chamber and an outer shell or jacket. A vacuum is created in this demarcated space, using a vacuum pump which is employed prior to, and then intermittently throughout the sterilization cycle. Steam can also be injected into this space, which helps to keep the steam that is injected into the sterilizer hot for as long a time as possible. The main drawback of this approach is that the sterilization cycle is extended by the amount of time needed to pump down the insulating space, which can add a significant delay. Other drawbacks of such a system are that a separate vacuum pump is required, with adds expense to the autoclave and adds the potential for mechanical breakdown. The noise created by the vacuum pump as well as the space required for the pump and steam generator precludes a table top autoclave design. Another drawback to the system is that it relies on the chamber warm-up coils to keep the chamber in a semi-ready state between sterilization cycles, thus expending electricity when the unit is essentially not in use. Since this type of sterilizer requires steam to be injected into the chamber, the chamber wall must be more than 212 degrees Fahrenheit (100 degrees Centigrade) so that the steam does not condense on the chamber wall when injected into the chamber. Still another drawback to the system is that the door to the sterilizer chamber is not surrounded by a vacuum, and provides for a large area of heat loss.

It has long been known that excellent insulating capability can be obtained by providing a vacuum between two members. A common example this principle is the vacuum flask container, where the temperature of the contents of a flask remains reasonably stable when the outer wall of the chamber is enclosed by a spaced, secondary surrounding wall. A fixed vacuum, created in the manufacturing process of the container exists in the space defined by these two walls. As the walls of such a vacuum flask are subjected to substantial forces due to the presence of a vacuum, these flasks are typically cylindrical in shape, and the walls of the flask, and in particular the outer wall, must be strong enough to resist these forces. The benefit if a mirrored or highly polished inner surfaces of a vacuum chamber are also well known, as they reflect back the radiated heat.

It has also long been known that when water evaporates from the bulb of a wet-bulb thermometer, latent heat is lost. Given a wet bulb and a dry bulb thermometer situated in a closed chamber, any presence of air in the closed chamber would result in a lower temperature reading for the wet bulb thermometer due to evaporation. The temperature at which steam will completely saturate a closed environment is dependent upon pressure. Notwithstanding changes in pressure, a closed environment is said to be saturated when there is no longer heat transfer between a wet bulb and a dry bulb thermometer, and an equilibrium exists within the chamber. Recent advances in hygrometer technology provide for simple yet precise electronic sensors that accurately measure both humidity and temperature.

Another well-known problem associated with steam sterilization is the corrosive effect the steam has on certain carbon steel items being sterilized. The present inventor has previously described (U.S. Pat. No. 5,707,553) a method wherein a modified distilled water solution used in an autoclave helped to prevent steam induced corrosion of such instruments. Other well-known methods of corrosion prevention have included the application of a vacuum pump to the sterilization chamber at the conclusion of a sterilization cycle to purge all moisture from the chamber.

The present invention describes a novel and effective design of an autoclave sterilizer to ensure that the autoclave chamber is filled with saturated steam, thus devoid of air. It also describes a novel application of a self-contained fixed vacuum that insulates the entire sterilizer chamber, and uses a proportional control thermostat, thus overcoming the aforementioned difficulties of heat loss and precise temperature control encountered with autoclave sterilizers.

### SUMMARY OF THE INVENTION

In accordance with the general principles of the invention, a table top steam sterilizer, commonly known as an autoclave is herein described. In accordance with a first aspect of the invention, a single heating coil circumferentially surrounds the outer surface of the sterilizer chamber. The volume of the chamber is less than two cubic feet (56 liters). A spaced secondary outer wall of the chamber demarcates an inter-wall space of approximately ten millimeters, into which a fixed vacuum is introduced on manufacture. This vacuum prevents heat loss through the walls of the sterilizer. The walls are composed of stainless steel approximately 0.3 millimeters in thickness. The inner surfaces of both walls are highly polished to aid in heat reflection and heat conservation. The door of the sterilizer chamber, although not directly heated, is spaced, double layered and fixed vacuum insulated in the same manner.

In accordance with another aspect of the invention, an electronic temperature/humidity sensor is placed in the lowermost, rearmost location in the sterilizer chamber. The sensor is connected to a microprocessor. When a sterilization cycle is started, a measured amount of water is introduced into the preheated chamber. As steam forms inside the chamber, temperature and pressure increase. As more steam is produced, it displaces air down and out of the chamber through a venting air bellows. Once the sensor determines that sterilization chamber is fully steam saturated, and the temperature of the chamber is at a programmed minimum, the microprocessor then closes the air vent. This causes the temperature and pressure within the chamber to continue to rise until the desired sterilization temperature is reached.

In accordance with another aspect of the invention, the heating coil surrounding the chamber wall is controlled by a proportional controller. The proportional controller performs better than the on-off power controllers typically found in sterilizer heaters. Using a proportional controller, chamber overheating is prevented as power to the heating element is applied in proportion to the difference in the sterilization set point and the actual sterilizer chamber temperature as measured by the microprocessor-controlled humidity/temperature probe. In a more advanced implementation the controller is programmed to execute a combination of a PID (Proportional—Integral—Derivative) algorithm as well as a predictive (also known as feed-forward) algorithm. As heat loss through the sterilizer chamber wall is significantly prevented, the ability of the proportional controller to precisely deliver the correct amount of power to the circumferential heating coil of the chamber is enhanced, thus preventing potentially destructive temperature overshoots within the chamber.

A further aspect of the invention involves the use of a venturi mechanism to evacuate moist air form the sterilizer chamber at the end of a cycle. Compressed air, readily available in, for example, dental offices where autoclaves are widely used, is passed through the venturi for a period of about twenty seconds. As the air moves across the constriction in the venturi, a vacuum is created, thus drawing moist air from the chamber and ensures a dry environment for the contents of the chamber. The contents can then be removed at the convenience of the autoclave operator.

Other features and advantages of the invention will be apparent to those familiar with steam sterilizers from the following detailed preferred embodiments of the invention and from the claims.

### BRIEF DESCRIPTION OF THE DRAWINGS

FIG. 1 is a sequence diagram for the invention.

FIG. 2 is a simplified cross section of a sterilizer according to the invention.

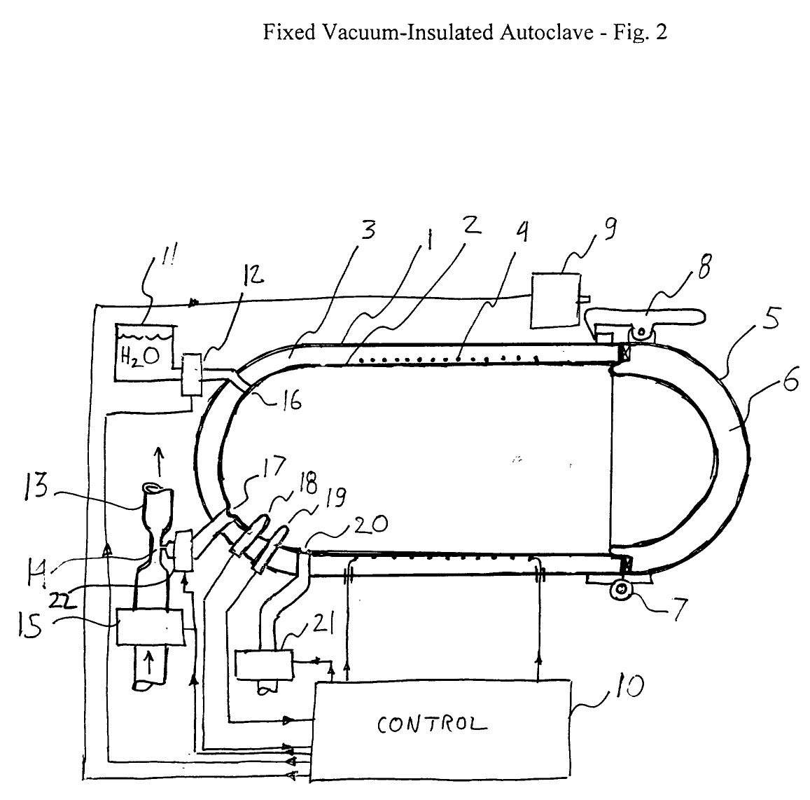

### DETAILED DESCRIPTION OF THE PREFERRED EMBODIMENT

In the preferred embodiment a sterilizer having an internal volume of between one quarter to two cubic feet (five to 60 liters) comprises of an outside cylindrical enclosure 1, an inner cylinder 2 with a permanently sealed vacuum space 3 of about 10 millimeters. The access door 5 is dome shaped, as is the end of cylinder 1 and has its own vacuum chamber 6. The main cylinders 1 and 2 as well as the access door 5 are made of stainless steel, with the inner surfaces, facing the vacuum, polished to a mirror-like finish to minimize radiation heat losses. The door is hinged by hinge 7 and latched by latch 8 having an optional electromechanical safety device 9 to prevent opening of the door prior to the end of the cycle. The inner wall 2 is made of thin metal, in the order of 0.3 to 1 millimeter in thickness, to reduce heating time. Heating is provided by heating elements 4 wound around the inner cylinder 2. A controller 10 controls the heating as well as the water inlet valve 12 connecting water supply 11 to cylinder 2 via port 16. The controller also controls a drain port 20 via valve 21 and a vacuum port 17 via valves 22 and 15. Valve 15 allows compressed air, normally available at dentist's offices, to create a vacuum by using a venturi device 14. Drain port 20 is installed at the lowest point in cylinder 2 in order to allow complete draining. The process is monitored via a temperature and humidity sensor 18 and a pressure sensor 19. Such sensors are commercially available from vendors such as Omega (web address: www.omega.com). The water supply 11 can be a container delivering a metered amount of demineralized water or any other supply of demineralized water.

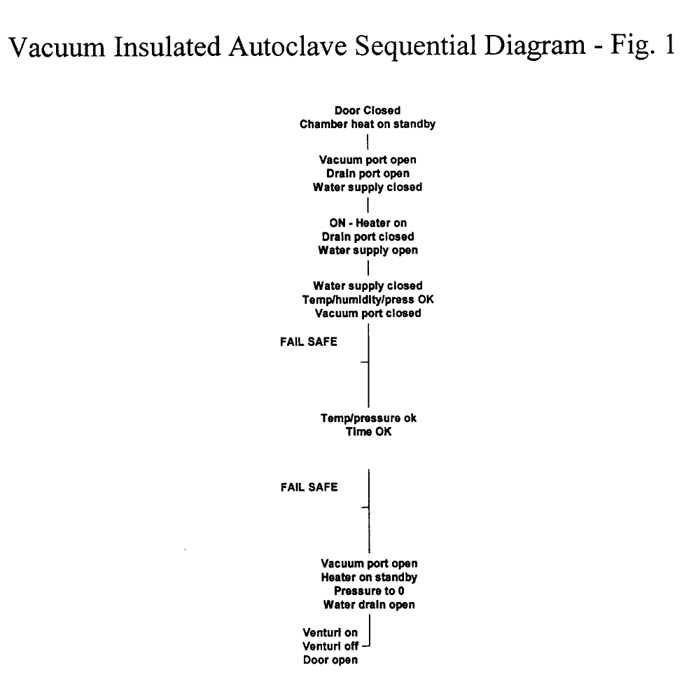

To begin a sterilization cycle, the vacuum insulated chamber door 5 is closed and locked by latch 8. The appropriate sterilization cycle is then chosen. The steps of the cycle are shown in FIG. 1. Typically, sterilization cycles could be 250 degrees Fahrenheit (121 degrees Centigrade) at about 15 pounds per square inch (1.03 kilobar) of pressure for 15 minutes, and 270 degrees Fahrenheit (132 degrees Centigrade) for three minutes at about 30 pounds per square inch (2.06 kilobar). Upon initiation of the sterilization cycle, the drain port 20 is closed and the water inlet valve 12 is opened. The chamber wall is heated to above 212 degrees Fahrenheit (100 degrees Centigrade) to avoid water condensation on the walls. A premeasured amount of demineralized water is released at the rear of the chamber from the water supply 11. The vacuum port valve 22 is open. When the pressure sensor attains a predetermined value, and the humidity/temperature probe determines that the air in the chamber is totally saturated, the vacuum port valve 22 is closed. Pressure and temperature is allowed to rise until the temperature has reached the desired level. The temperature is maintained at this level for the desired amount of time. The presence of an insulating vacuum and a proportional controller will allow temperature fluctuation inside the chamber of no greater than +/− two degrees Fahrenheit (+/− one degree Centigrade). Once the sterilization time has been reached, the air vent valve 22 is opened, and pressure inside the chamber is released. When the pressure in the chamber reaches near zero, the drain port valve 21 is opened, allowing residual water in the chamber to drain. This valve is then closed after a 30 second time interval. The venturi valve 15 is then opened, allowing compressed air to flow through the venturi, thus creating a vacuum inside the chamber. This valve is kept open for 30 seconds. As air moves through the venturi and the chamber vacuum is created, any residual moisture inside the chamber is evacuated through the venturi end escapes through the vacuum port 17. At this point the sterilization chamber door can be opened, and the articles inside the chamber are dry and sterilized. An electromechanical interlock 9 can be provided which prevents opening the door before the end of the cycle. Such interlocks are well known in the art. The electronic control 10 can be implemented in any of the ways known in the art, preferably a microprocessor or a PLC (Programmable Logic Controller). A small display keeps the user informed about the progress of the process.

The invention has been described presenting specific embodiments. However, it will be recognized by those familiar with the art of steam sterilization, various modifications and substitutions may be made within the scope of the invention as defined by the appended claims.

What is claimed is:

1. A steam sterilization method comprising of the steps of:
placing objects to be sterilized in a double walled container having the cavity between the two walls evacuated and permanently sealed, and controlling the temperature inside the container to achieve complete sterilization.

2. A steam sterilization method comprising of the steps of:
placing objects to be sterilized in a double walled container having the cavity between the two walls evacuated and permanently sealed; and
controlling the temperature inside the container to achieve complete sterilization, said temperature control using a proportional control method.

3. A steam sterilization method comprising of the steps of:
placing objects to be sterilized in a double walled container having the cavity between the two walls evacuated and permanently sealed, partially evacuating said container using a venturi for said evacuation by use of compressed air and a venturi device; and
controlling the temperature inside the container to achieve complete sterilization.

4. A steam sterilizer comprising of a double walled container wherein the cavity between two said walls has been permanently evacuated and sealed.
